(19) United States
(12) Patent Application Publication
(10) Pub. No.: US 2026/0144677 A1
(43) Pub. Date: May 28, 2026

(54) SURGICAL SYSTEMS AND METHODS FOR ANTERIOR GONIOTOMY

(71) Applicants: RAICO International LLC, Westmont, IL (US); INNOVATIVE GLAUCOMA CONSULTING LLC, Dallas, TX (US); EYEINNOVATE LLC, Dallas, TX (US)

(72) Inventors: Ronald L. Fellman, Dallas, TX (US); Davinder S. Grover, Dallas, TX (US); Sebastian S. Grover, Dallas, TX (US); Ravi Nallakrishnan, Willowbrook, IL (US)

(21) Appl. No.: 19/489,500

(22) PCT Filed: Jun. 5, 2024
(86) PCT No.: PCT/US2024/032543

§ 371 Date: Dec. 2, 2025

Related U.S. Application Data
(60) Provisional application No. 63/471,789, filed on Jun. 8, 2023.

(51) Int. Cl. A61F 9/007; A61B 3/10; A61F 9/008

(52) U.S. Cl. CPC A61F 9/00781; A61B 3/102; A61F 9/00736; A61F 9/008; A61F 2009/00851; A61F 2009/00872; A61F 2009/00891

(57) ABSTRACT

A surgical system for performing a goniotomy procedure includes a means for creating a trabecular leaflet at Schwalbe's line in an eye, wherein the trabecular leaflet is characterized by an opening angle of between about 4 degrees and about 45 degrees defined between the trabecular leaflet and the outer wall of the Schlemm's Canal. The means may include, but is not limited to, a handheld microsurgical cutting instrument having at least one sharpened edge, an invasive or non-invasive laser cutting instrument, a vibratory cutting instrument, or a thermal cutting instrument. In one form, the trabecular leaflet is further characterized as having an arcuate, concave portion facing the outer wall of the Schlemm's Canal.

### PRIORITY

The present application claims priority from U.S. Provisional Ser. No. 63/471,789, filed on Jun. 8, 2024, the entire contents of which is incorporated herein by reference.

### FIELD OF THE INVENTION

The present invention relates to surgical systems and methods for performing ophthalmological procedures for treatment of eye diseases, such as glaucoma, and more particularly to surgical systems and methods to facilitate an anterior goniotomy procedure.

### BACKGROUND OF THE INVENTION

Goniotomy was initially described as a surgical procedure to treat congenital and developmental glaucomas that are caused by a developmental abnormality in the trabecular outflow system. As a result of this abnormality, the trabecular meshwork itself becomes thicker and these changes lead to elevated intraocular pressure that can damage the internal structures of the eye, including the optic nerve leading to the development of glaucoma. Later, goniotomy was found to lower eye pressure in adults.

The purpose of a goniotomy is to selectively cleave the abnormal trabecular tissue in order to improve the flow of aqueous from the eye, which in turn lowers the intraocular pressure (IOP). Lowering the IOP helps to stabilize the enlargement of the cornea and the distension and stretching of the eye that often occur in congenital/developmental glaucoma. Importantly, once the aqueous outflow improves, damage to the optic nerve is halted and may be reversed. The patient's visual acuity may improve after surgery. In adults, goniotomy cleaves open diseased outflow tissues similar to its effect of decreasing outflow resistance in the childhood glaucomas.

The goniotomy procedure can restore normal drainage of aqueous humor from the eye by cleaving a segment of the trabecular meshwork, thus allowing the aqueous humor to drain through the open area from which the strip of trabecular meshwork has been cleaved. The goniotomy procedure and certain prior art instruments useable to perform such procedure are described in U.S. Pat. No. 6,979,328, hereby incorporated by reference in its entirety.

U.S. Pat. No. 4,846,172 A and 6,241,721 B1 teach invasive laser ablation instruments and methods for forming artificial passageways in the trabecular meshwork while U.S. Pat. No. 6,059,772 A, U.S. Patent Application Publication No. US 2020/0330266 A1, and International Patent Application Publication No. WO 2020/018243 A1 teach non-invasive laser ablation instruments and methods for forming artificial passageways in the trabecular meshwork, and all of said prior art documents are hereby incorporated by reference in their entireties as if fully set forth herein. Generally, these prior art disclosures taught the boring of artificial holes or passageways into or through the trabecular meshwork to improve flow therethrough with or without the implantation of a shunt or other artificial passageway in the eye.

At present there remains a need in the art for the development of improved, easy to use, inexpensive, minimally invasive systems and methods to perform the goniotomy procedure, or other similar procedures, to reduce intraocular pressure with long-lasting effects to reduce or eliminate follow-up procedures and/or to reduce the number of intraocular pressure reducing medications prescribed to a patient.

### Brief Summary of the Invention

In accordance with one broad form of the present invention, a goniotomy surgical system is disclosed and includes a means for creating a trabecular leaflet at, or just posterior to, Schwalbe's line in an eye. The trabecular leaflet is characterized by an opening angle of between about 4 degrees and about 45 degrees defined between the trabecular leaflet and the outer wall of the Schlemm's Canal, when viewed in a vertical cross-section containing the optical axis of the eye and extending through the incision as illustrated in FIGS. 13E-13F. Preferably, the trabecular leaflet is characterized by an opening angle of about 10 degrees defined between the trabecular leaflet and the outer wall of the Schlemm's Canal.

In one form of the present invention, the trabecular leaflet formed by the system is further characterized as having an arcuate, concave portion facing the outer wall of the Schlemm's Canal. Preferably, the arcuate, concave portion of the trabecular leaflet defines a central angle of between 30 degrees and 60 degrees.

According to another form of the present invention, the trabecular leaflet formed by the system is further characterized as having an arcuate, concave portion of defining a ratio of arc length to radius of between about 0.5 and 0.9.

In one preferred form of the present invention, the means for creating a trabecular leaflet is a handheld microsurgical cutting instrument having at least one sharpened edge.

In another form of the present invention, the means for creating a trabecular leaflet is an invasive laser cutting instrument for being inserted into the anterior chamber of the eye and having at least one laser source.

In another form of the present invention, the means for creating a trabecular leaflet is a non-invasive laser cutting instrument configured to form the trabecular leaflet from outside of the cornea of the eye and having at least one laser source.

According to another form of the present invention, the means for creating a trabecular leaflet is a vibratory cutting instrument configured to form the trabecular leaflet and having at least one vibratory cutting edge.

According to another form of the present invention, the means for creating a trabecular leaflet is a thermal or cryo cutting instrument configured to form the trabecular leaflet and having at least one thermal or cryo cutting tip.

According to another form of the present invention, the means for creating a trabecular leaflet is a pulsing water jet cutting instrument configured to form the trabecular leaflet and having at least one hydrodissection cutting tip.

According to another form of the present invention, the surgical system includes one of an internal reservoir of an irrigating fluid or a connection to an external reservoir of irrigating fluid and means for selectively applying the irrigating fluid to the trabecular leaflet.

In accordance with one broad form of the present invention, a method of using a goniotomy surgical system to treat the trabecular meshwork of the eye is disclosed. The method includes the step of obtaining a goniotomy surgical system including a means for creating a trabecular leaflet at, or just posterior to, Schwalbe's line in an eye. The trabecular leaflet is characterized by an opening angle of between about 4 degrees and about 45 degrees as defined between the trabecular leaflet and the outer wall of the Schlemm's Canal. The method includes the step of creating or forming a trabecular leaflet in the eye with the means, wherein the trabecular leaflet is characterized by an opening angle of between about 4 degrees and about 45 degrees as defined between the trabecular leaflet and the outer wall of the Schlemm's Canal. Preferably, the trabecular leaflet formed by the method is characterized by an opening angle of about 10 degrees defined between the trabecular leaflet and the outer wall of the Schlemm's Canal.

In one form of the present invention, the trabecular leaflet formed by the method is characterized by having an arcuate, concave portion facing the outer wall of the Schlemm's Canal. Preferably, the arcuate, concave portion of the trabecular leaflet defines a central angle of between about 30 degrees and about 60 degrees.

According to another form of the present invention, the trabecular leaflet formed by the method is characterized by having an arcuate, concave portion defining a ratio of arc length to radius of between about 0.5 and about 0.9.

In one preferred form of the present invention, the means for creating the trabecular leaflet in the method is a handheld microsurgical cutting instrument having at least one sharpened edge.

In one preferred form of the present invention, the means for creating the trabecular leaflet in the method is an invasive laser cutting instrument for being inserted into the anterior chamber of the eye and having at least one laser source.

In another form of the present invention, the means for creating the trabecular leaflet in the method is a non-invasive laser cutting instrument configured to form the trabecular leaflet from outside of the cornea of the eye and having at least one laser source.

According to another form of the present invention, the means for creating the trabecular leaflet in the method is a vibratory cutting instrument configured to form the trabecular leaflet and having at least one vibratory cutting edge.

According to another form of the present invention, the means for creating the trabecular leaflet in the method is a thermal or cryo cutting instrument configured to form the trabecular leaflet and having at least one thermal or cryo cutting tip.

According to another form of the present invention, the surgical system used in the method includes one of an internal reservoir of an irrigating fluid or a connection to an external reservoir of irrigating fluid and means for selectively applying the irrigating fluid to the trabecular leaflet.

In one form of the present invention, the method step of creating a trabecular leaflet in the eye with the means further includes creating the trabecular leaflet between about 90 degrees and about 180 degrees around the periphery of the eye relative to the optical axis of the eye. More preferably, the trabecular leaflet is formed about 120 degrees around the periphery of the eye relative to the optical axis of the eye.

In another form of the present invention, the system further includes an imaging system having an optical coherence tomography beam configured to create an image of the eye. The means for creating a trabecular leaflet may be controlled based on the image of the eye.

### BRIEF DESCRIPTION OF THE DRAWINGS

In the accompanying drawings forming part of the specification, in which like numerals are employed to designate like parts throughout the same,

FIG. 1 is a left-side elevation view of a first embodiment of a surgical system according to the present invention, wherein the system has the form of a handheld cutting instrument;

FIG. 2 is a top plan view of the instrument of FIG. 1;

FIG. 3 is an isometric view, taken from above and the right side, of the instrument of FIG. 1;

FIG. 4 is an enlarged, isometric view, of the operative, distal tip portion of the instrument of FIG. 3;

FIG. 5 is a greatly enlarged, fragmentary, left-side elevation view of the distal tip portion of the instrument shown in FIG. 1;

FIG. 6 is a greatly enlarged, fragmentary, top plan view of the distal tip portion of the instrument shown in FIG. 1;

FIG. 7 is a left-side elevation view of a second embodiment of a surgical system according to the present invention, wherein the system has the form of a handheld cutting instrument;

FIG. 8 is a top plan view of the instrument of FIG. 7;

FIG. 9 is an isometric view, taken from above and the right side, of the instrument of FIG. 7;

FIG. 10 is an enlarged, isometric view, of the distal portion of the instrument of FIG. 7;

FIG. 11 is a greatly enlarged, fragmentary, left-side elevation view of the distal tip portion of the instrument shown in FIG. 7;

FIG. 12 is a greatly enlarged, fragmentary, top plan view of the distal tip portion of the instrument shown in FIG. 7;

FIG. 12A is a greatly enlarged, fragmentary, top plan view, of a distal portion of a variation of the second embodiment of a surgical system according to the present invention, wherein the system has the form of a handheld cutting instrument;

FIG. 12B is a greatly enlarged, isometric view from above, of the distal portion of the instrument shown in FIG. 12A;

FIG. 13A is an optical coherence tomography image of an eye subsequent to an operation with an instrument according to the present invention, and FIG. 13A shows the formation of a trabecular leaflet or flap at 1-month post operation;

FIG. 13B is an optical coherence tomography image of an eye subsequent to an operation with an instrument according to the present invention, and FIG. 13B shows the formation of a trabecular leaflet or flap at 12-months post operation;

FIG. 13C is an optical coherence tomography image of an eye subsequent to an operation with an instrument according to the present invention, and FIG. 13C shows the formation of a trabecular leaflet or flap at 17-months post operation;

FIG. 13D is another optical coherence tomography image of an eye subsequent to an operation with an instrument according to the present invention, and FIG. 13D shows the classic formation of a trabecular leaflet or flap;

FIG. 13E is a greatly enlarged portion of the image of the eye of FIG. 13D, and FIG. 13E shows the leaflet in greater detail;

FIG. 13F is a greatly enlarged portion of the image of the eye of FIG. 13D, and FIG. 13F shows the leaflet in greater detail;

FIG. 13G is a diagrammatic, simplified cross-sectional view of the eye subsequent to an operation with an instrument according to the present invention showing the flow structures behind the trabecular meshwork; and

FIG. 14 is a greatly simplified, diagrammatic view of another embodiment of a surgical system according to the present invention, wherein the system may be configured as an invasive or non-invasive laser instrument, vibratory or pulsing cutting instrument, cautery or thermal cutting instrument, trephination surgical instrument, or cryo surgical instrument.

### DETAILED DESCRIPTION OF THE PREFERRED EMBODIMENTS

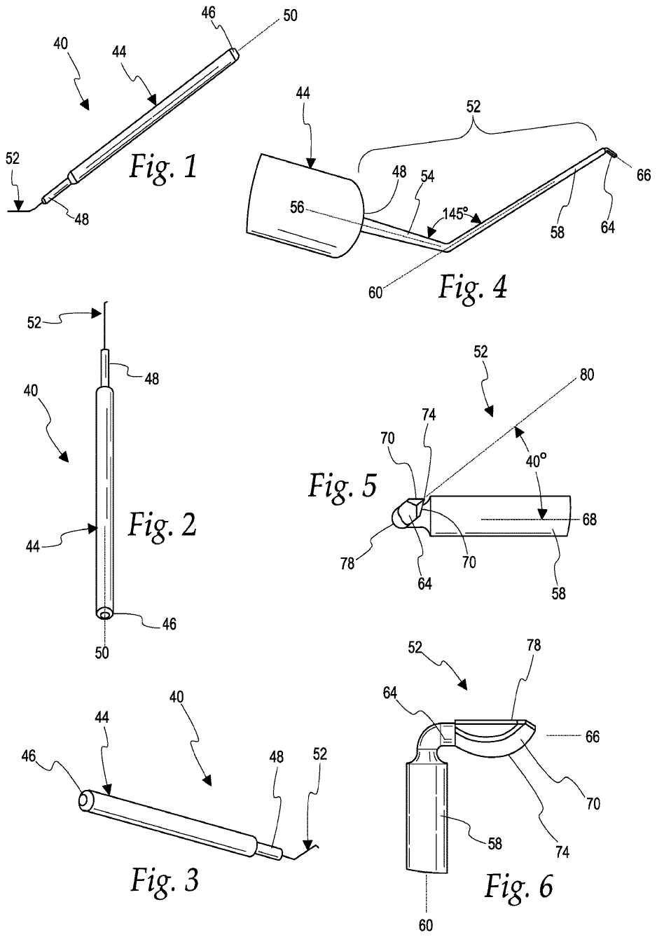

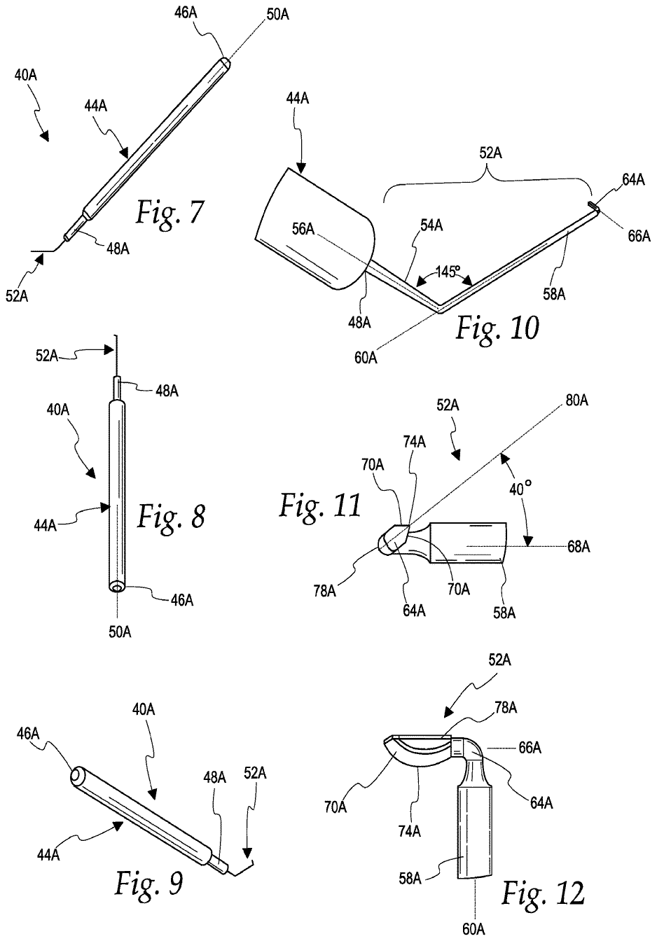

With reference to FIGS. 1-6, in accordance with a first illustrated embodiment of the present invention, a present goniotomy surgical system that is configured as a hand-held, manually operated cutting instrument 40 which includes a hand piece or hand grip 44 for being gripped by a user of the instrument 40. The grip 44 has an elongated configuration, including a proximal end 46 and a distal end 48 defining a hand grip axis 50 extending therebetween. FIGS. 1-6 show a first variation of the instrument 40 for use as a right-angled instrument. FIGS. 7-12 show a left-angled variation of the instrument 40A, which will be discussed in greater detail below. It is further contemplated that this embodiment of the instrument 40 may also be configured as a non-angled, straight instrument. Each of these variations of the instrument 40 and 40A are configured to contact specific angles, arcs, or portions of the trabecular meshwork of a patient's eyes, as will be discussed in detail below. The hand grip 44 can be provided with either a rounded or a flattened configuration and is preferably knurled for ease of grip. Furthermore, the hand grip 44 may be a cannula hub or fitting with means for being attached to a larger machine, irrigation system, or commercial irrigating handpiece (e.g., screw threading, snap-fit connection, luer lock connection, friction fit, locked, etc.). The numbered features of the embodiments of the instruments 40 and 40A illustrated and discussed herein are designated generally with a number where such features are analogous in structure and function.

With reference now to FIG. 4, the instrument 40 includes a tip 52 that extends, either directly from the distal end 48 of the hand grip 44, or indirectly from the distal end 48 of the hand grip 44 via one or more straight or angled shank portions depending on the right, left, or straight designed use of the instrument 40.

In the illustrated first preferred embodiment of the instrument 40, the tip 52 includes a base portion 54 that extends from the distal end 48 of the hand grip 44 and defines a base portion axis 56 through its geometric center. The base portion axis 56 is substantially parallel to, and coincident with, the central axis 50 of the hand grip 44. The tip 52 further includes an intermediate portion 58 extending from the base portion 54 along an intermediate portion axis 60 that is transverse or angled relative to the base portion axis 56. Preferably, the intermediate portion axis 60 is angled between about 130 degrees and 160 degrees relative to the base portion axis 56, and more preferably angled about 145 degrees. The tip 52 includes a terminal portion 64 extending from the intermediate portion 58 along a terminal portion axis 66 that is transverse to the intermediate portion axis 60. Preferably, the terminal portion axis 66 is angled between about 90 degrees and about 140 degrees relative to the intermediate portion axis 60, and more preferably is angled about 120 degrees. The terminal portion 64 includes cutting means for creating a trabecular leaflet at, or just minimally posterior to or below, Schwalbe's line in an eye. The inventors have found that increasing the angle between axes 66 and 60 from about 90 degrees to about 120 degrees greatly improves the user's ability to perform the surgical operation of creating the leaflet.

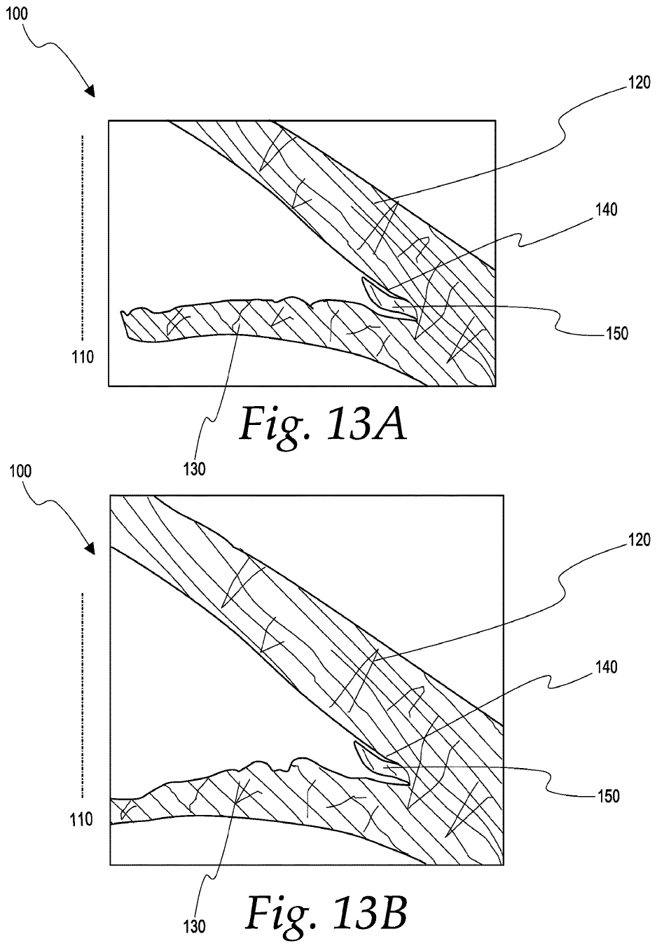

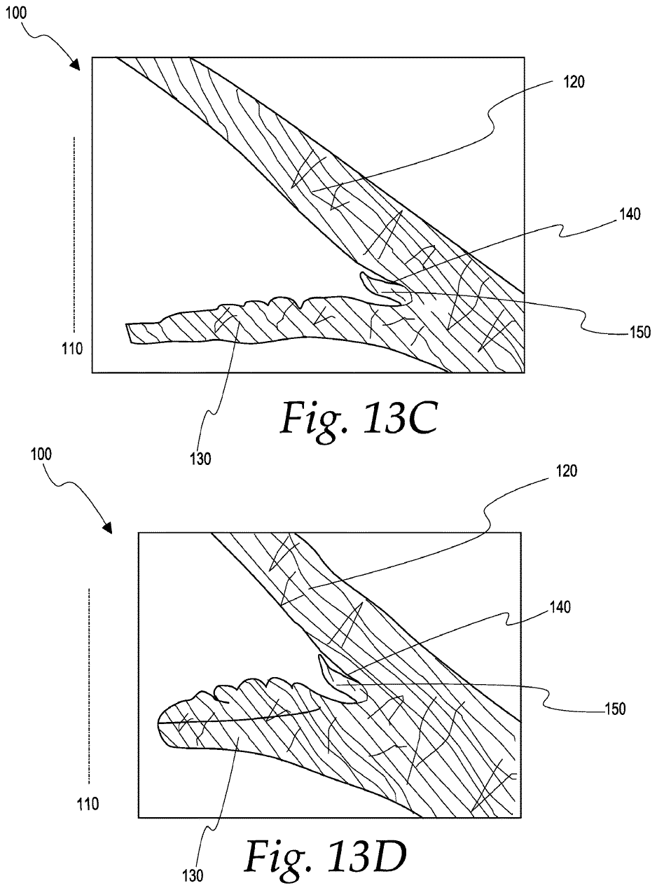

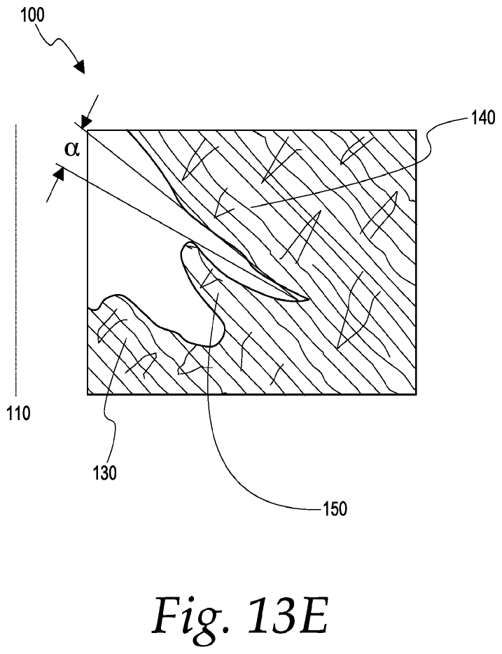

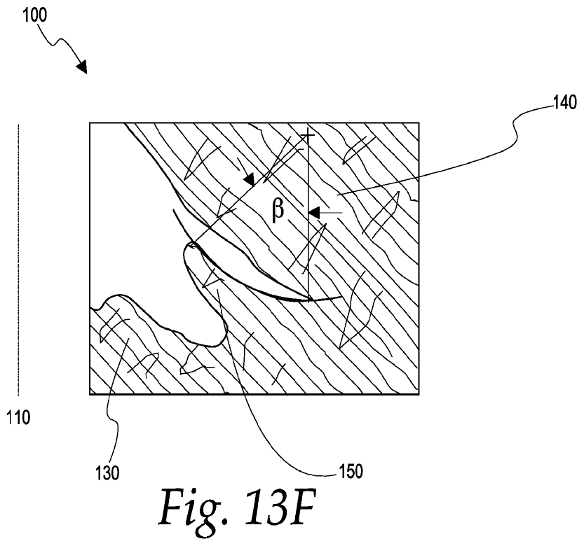

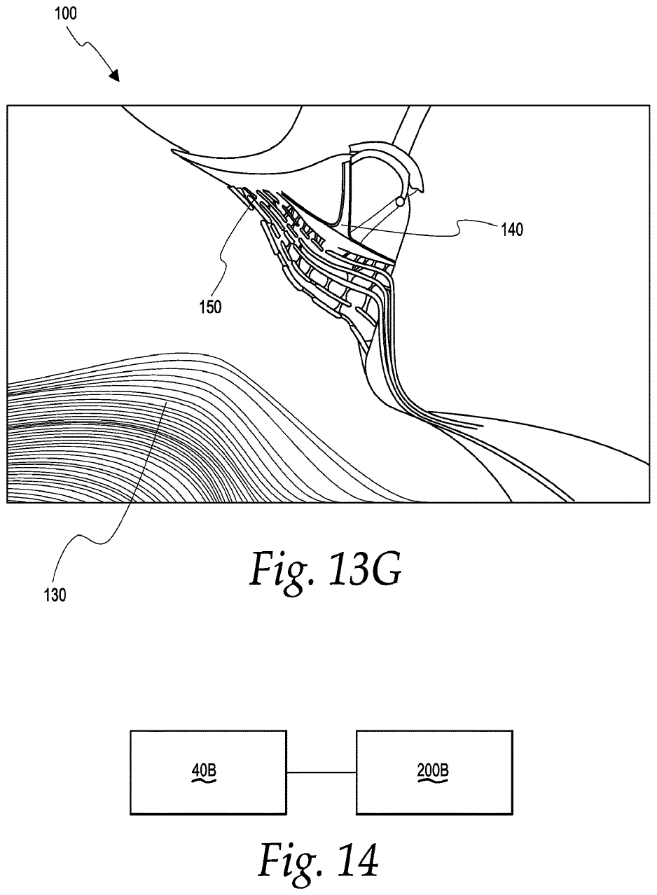

With reference to FIGS. 13A-13F, a series of optical coherence tomography images of an eye 100 that has been operated upon by the system in the form of the instrument 40 is shown. Directions referenced herein are generally taken relative to the optical axis 110 (or a line parallel thereto) of such an eye 100, wherein the anterior direction is axially upwardly in FIGS. 13A-13F away from the optic or lens of the eye, and the posterior direction is axially downwardly in FIGS. 13A-13F toward the optic of the eye. FIGS. 13A-13F show the cornea 120, iris 130, and the opening of Schlemm's canal 140 of the eye 100. The inventors have found that the instrument 40 disclosed herein is advantageously configured to safely and efficiently incise the trabecular tissue directly below Schwalbe's line to allow the separated trabecular meshwork or tissue 150 to drop away from the cornea 120 in the posterior direction toward the iris 130. The trabecular leaflet or flap 150, which extends an angle or arc around the optical axis 110, may improve flow through the Schlemm's canal 140 to lower intraocular pressure and reduce the medications required by a patient with glaucoma. FIG. 13A shows the eye 100 after 1 month from the date of the surgery with the instrument 40 of the present invention, FIG. 13B shows the same eye 100 after 12 months from the date of the surgery, and FIG. 13C shows the same eye 100 after 17 months from the date of the surgery. FIG. 13D shows a classic appearance of the trabecular vent in the eye 100 following an iVent surgery with the present invention. The acute angle of the trabecular corneal angle is classic for a post operative appearance of the vent created with the inventive technique. The appearance of this trabecular vent angle is distinctly different from the appearance of angles after other forms of goniotomy, GATT, OMNI, etc.

FIG. 13G is a diagrammatic, computer assisted drawing model of the post-operative eye 100 that has been operated upon by the system in the form of the instrument 40. FIG. 13G shows the iris 130, and the opening of Schlemm's canal 140 and valve structures behind the trabecular meshwork, and the inventive leaflet 150 in the post-operative eye 100.

With reference now to FIGS. 5 and 6, the preferred cutting means of the instrument 40 has the form of a pair of sloping, arcuate cutting surfaces 70 that join to define an arcuate cutting edge 74. The cutting surfaces 70 are preferably semi-circular and define a bevel terminating rearwardly to face the hand grip 44 of the instrument 40 when gripped by a user. As can be seen in FIG. 5, the instrument 40 defines a plane 68 that contains the axes 60 and 66 of the intermediate tip portion 58 and the terminal portion 64 of the tip 52. The arcuate cutting edge 74 defines a central edge axis 80 that is angled between about 30 and 50 degrees, and more preferably about 40 degrees relative to the plane 68. The distal most end of the terminal portion 64, relative to the hand grip 44, is a blunted surface 78 that is opposite the arcuate cutting edge 74. The blunted surface 78 is the furthest portion of the tip 52 away from the hand grip 44.

With reference to FIG. 5, the terminal portion 64 of the tip 52 preferably has a width of about 0.1 mm.

With reference to FIG. 6, the terminal portion 64 of the tip 52 preferably has an operative blade length of about 0.30 mm, along the terminal portion axis 66 and a blade width of about 0.15 mm in the direction normal to the axis 66.

Other configurations of the cutting edge of the surgical system in the form of a manually-operated cutting instrument are contemplated.

The inventors of the present invention believe that the embodiments of the instrument discussed herein may advantageously permit the surgeon to improve flow through the outflow tissues by disinserting the trabecular meshwork from Schwalbe's line and simultaneously preserving the valve like function inherent to the outflow system. The instruments herein may lower costs for the glaucoma procedure compared to existing surgical instruments with expensive handpieces, and may improve surgical outcomes by reducing the time of the procedure compared to surgeries performed with prior art devices. The instruments disclosed herein may be easier to use by a surgeon, cost less than leading prior art devices on the market, and/or reduce or eliminate post-operative visits to the surgeon by the patient.

The present invention facilitates the creation of a unique incision into the trabecular tissue compared to all other currently know methods for opening the trabecular meshwork, including but not limited to Kahook Dual Blade, OMNI 360 Surgical System, iStent inject, Hydrus, GATT, Hemi-GATT and Tanito Goniotomy. An advantage of the presentinvention include the precise and selective opening of the anterior most aspect of the trabecular meshwork, essentially just posterior to Schwalbe's line. This selective incision of the trabecular meshwork allows for the cleavage of trabecular meshwork tissue with minimal disruption of the existing intracanalicular valvular system. The cleaved trabecular meshwork is detached from Schwalbe's line, but remains attached to the scleral spur. Depending on the elasticity ofthe leaflet and other factors including gravity and postoperative healing, the leaflet remains open to varying degrees. This selective anterior cleavage of the trabecular meshwork protects disruption of the microscopic support structures in the angle and theoretically preserves the pump/valve function within Schlemm's canal.

A further advantage of this procedure is to minimize trauma and preserve the valve system unlike the Kahook Dual Blade, ITrack, Omni 360, GATT prior art, Tanito, Espaillat, trabeculotomy or ABIC procedures. With minimal disruption of the microscopic support structures within Schlemm's canal in the present invention, the potential for reflux of blood due to hypotony is minimized (given that the valve structures remain essentially intact). Additionally, because there is not device or instrument that rubs along or disrupts the back wall of Schlemm's canal, this device also minimizes the risk of disrupting the endothelium lining of Schlemm's canal, any associated vascularized tissue and any of the microscopic intracanalicular canal structures such as valves and microtubes. Also, depending on the degree of glaucomatous disease and valvulardysfunction, the specific cleavage plane created in the anterior trabecular meshwork may allow for a stretching of the intracanalicular valve system with improvement in its function. Further, the unique design of the device allows for maintenance of the anterior chamber.

The inventors have termed the unique method of the subject invention interventional Valve Enhancing Trabeculotomy or “iVent”. The iVent makes a precise cleavage in the anterior aspect of the trabecular meshwork. Care is taken to avoid the intracanalicular structures and valves when creating this cleavage plane. By specifically cleaving the anterior trabecular meshwork a vent is created.

In regard to traction, by just incising the trabecular meshwork, the anterior attachment is released, and the posterior aspect of the trabecular meshwork shifts away from the anterior insertion into the corneal/scleral inner wall. The movement of the anterior trabecular meshwork from its insertion site allows for traction on the intracanalicular structures and enhancement of canalicular valve function. By enhancing the pump/valve mechanism, the IOP is lowered through improvement of the traditional outflow pathway. This mechanism of enhancement is specifically unique to iVent.

The iVent is different from other anterior segment angle surgeries as it spares disruption of the intracanalicular structures. The enhancement of the traditional outflow pathway may be considered similar to the enhancement seen after such procedures like selective laser trabeculoplasty or SLT, which also has the potential to enhance or rejuvenate an eye's outflow pathway. The cellular stimulation created by the iVent will also improve the pump mechanism of the intracanalicular valves. Additionally, because the trabecular meshwork is being cleaved and not incised and cored out (as is other type of angle surgeries) the iVent is less disruptive and results in a lower degree of acute intraocular inflammation. A significant population of unique cells with stem cell properties reside at Schwalbe's line as shown by (1) Raviola, G. Schwalbe's line's cells: a new cell type in the trabecular meshwork of Macaca mulatta. Invest Ophthalmol Vis Sci.m1982:22:45-56 and (2) Braunger B M, Ademoglu B, Koschade S E, at al., Identification of adult stem cells in Schwalbe's line region of the primate eye. IOVS 2014;55:7499-7507, which are both incorporated herein by reference in their entireties. These adult stem cells, known as Schwalbe's line cells, may compensate for the loss of trabecular meshwork cells associated with glaucoma. These cells may be directly stimulated by iVent to provide a population of pluripotent stem cells that are capable of differentiating into outflow cells that enhance the physiology of outflow. Cytokine release and other factors related to favorable wound healing from the iVent procedure likely stimulate these stem cells to provide a population of cells to improve outflow.

Importantly, the iVent does not involve implanting a foreign body and as such, there is no concern of displacement of a stent or object. There is no concern of erosion or malfunction with the present invention as with an implant. Given the lack of an implant and the minimally traumatic nature of this technique, the iVent is minimally traumatic to the corneal endothelial cells.

It is presently believed that IVent has a lower risk of abnormal wound healing as a result of being less destructive to the trabecular meshwork, as compared to the other prior art techniques mentioned above. The iVent further does not remove valve and channels of the trabecular meshwork, nor does it obstruct or close down such channels.

The iVent surgery has been found by the inventors to lower the intraocular pressure to the low teens when the patient is on one, or on no medications (i.e., medicated drops). The outcomes the inventors are seeing with iVent appear to be distinctly different from the outcomes we have seen with KDB, Trabectome, OMNI, hydrus and istent or other angle based surgeries. These prior art mentioned surgeries tend to have a more modest IOP lowering that results in a postoperative IOP in the mid-teen range with the patient on 1-2 medications. The fact that the inventors are seeing significantly lower eye pressures following the iVent surgery speaks to the novel and unique nature of this surgery and how it specifically enhances the patient's natural outflow pathway through improving the function of the intracanalicular pump/valve mechanism.

It will be understood that the instruments disclosed herein may be formed in a variety of sizes for small incision glaucoma surgery or regular glaucoma surgery.

In one presently preferred method of use of the instrument 40 may be configured for use in incising the nasal angle or temporal angle of the right eye and/or the left eye. The instrument 40 may be inserted through an incision in the cornea of the operative eye. An arc of the trabecular meshwork at (or minimally posterior of) to Schwalbe's line is then engaged by the cutting means of the instrument to create the unique cleavage plane. The trabecular meshwork drops in the posterior direction toward the iris. A gonioprism may be used to view the trabecular meshwork as it is engaged by the instrument 40. Irrigation fluid can optionally be applied on-demand by the user of the instrument 40 in a reflux burst or jet pulse.

Cutting of the anterior trabecular meshwork is achieved by incising just below or at Schwalbe's line. Based on the specific clinical case, the surgeon may selectively incise 1-5 clock hours. Additionally, the surgeon may choose to incise one area, leave an island of untreated trabecular meshwork, and incise a subsequent area, to maximize canal opening but minimize tissue disruption. This technique creates a unique anterior cleavage plane with the advantages of allowing the trabecularshelf or tissue to remain open and minimize disruption of microscopic structures within the canal. In fact, one could make a small corneal incision in the nasal quadrant and treat the temporal angle 180 degrees, thus creating a 360 degree iVent.

The design of the instrument of the present invention precisely allows for alignment to Schwalbe's line without any difficult positional maneuver for making the incision up to the 6 clock hours (when seating temporal to the patient). This is done easily with the left & right symmetrically designed instruments. If the surgeon were to sit on the opposite side, they could potentially have access to the lateral 6 clock hours and in theory treat 360 degrees if desired, although this approach is not the primary intent of this instrument however the inventors have taken this approach in certain situations.

The inventors believe that the concept that the eye outflow system is a passive filter is outdated. Indeed, Dr. Jorge A. Alvarado found that severe alterations occur in the cellular component and in the entire trabecular meshwork during primary open-angle glaucoma and ageing. The same author demonstrates that trabecular meshwork endothelial cells regulate aqueous outflow by actively releasing enzymes and cytokines that, upon binding to Schlemm's canal endothelial cells, increase transendothelial flow thereby facilitating the egress of aqueous humour. Trabecular meshwork endothelial cells secrete these factors in response to stimuli such as mechanical stretching, laser irradiation, and pro-inflammatory cytokines.

The inventors of the present invention believe that the instruments and methods of the present invention could be very effective with potential longer lasting results.

It is believed that incising the trabecular meshwork just posterior to the location of Schwalbe's line allows for the creation of a unique trabecular flap, unlike any other created by the prior art instruments. This flap is hinged posteriorly at the scleral spur, however, the angle of opening for the flap below Schwalbe's is intended to preserve and possibly enhance the intracanalicular structures (i.e., valves, tubules, etc.). These structures are disrupted when a suture, filament or catheter of the prior art techniques are threaded through Schlemm's canal or when a trabecular shelf is parallel to the iris. However, when a specifically designed spatula that is angled, beveled, and curved is used to create a selective and precise anterior incision in the trabecular meshwork, these intracanalicular structures are preserved and protected. In fact, there is a very high chance that the trabecular meshwork flap being released at its most anterior insertion will provide some tension or stretch on the intra-canalicular valves, a valvulotasis termed by the inventors, and potentially enhance their function.

The inventors of the present invention have found that the leaflet 150 created by the present invention may be characterized in a number of aspects. A first aspect of the leaflet characterization, with reference to FIG. 13E, is the trabecular meshwork/outer wall of the Schlemm's Canal opening angle a. Preferably, the opening angle a is between about 4 degrees and about 45 degrees when viewed in cross-section in a vertical plane that that contains the optical axis 110 of the eye and extends through the incision as shown. More preferably, the opening angle a is about 10 degrees (+/−5 degrees).

A second aspect of the leaflet 150 characterization, with reference to FIG. 13F, is that the leaflet has an arcuate, concave portion or surface facing the outer wall of Schlemm's Canal. Preferably, the arcuate, concave portion may define a central angle β between about 30 degrees and about 60 degrees. More preferably, the central angle β is about 54 degrees. Furthermore, the arcuate, concave portion of the trabecular leaflet 150 defines a ratio of arc length to radius of between about 0.5 and about 0.95. More preferably, the arc length is about 286 microns, and the circle radius is 303.5 microns, with a ratio of 0.94.

Referring now to FIGS. 7-12, another embodiment of the present goniotomy surgical system invention, which is configured as a hand-held, manually operated cutting instrument is illustrated and designated as 40A. The numbered features of the instrument 40A are designated generally with the suffix letter “A” and are analogous to features of the aforementioned illustrated embodiment of the instrument 40 that share the same number (without the suffix letter “A”). The instrument 40A operates in an identical manner as described in detail above and has the same basic features of a hand grip 44A with a tip 52A including a base portion 54A that extends from the distal end 48A of the hand grip 44A and defines a base portion axis 56A through its geometric center. The base portion axis 56A is substantially parallel to, and coincident with, the central axis 50A of the hand grip 44A. The tip 52A further includes an intermediate portion 58A extending from the base portion 54A along an intermediate portion axis 60A that is transverse or angled relative to the base portion axis 56A. Preferably, the intermediate portion axis 60A is angled between about 130 degrees and 160 degrees relative to the base portion axis 56A, and more preferably angled about 145 degrees. It is further contemplated that this embodiment may be configured as a non-angled, straight instrument.

The tip 52A includes a terminal portion 64A extending from the intermediate portion 58A along a terminal portion axis 66A that is transverse to the intermediate portion axis 56A. Preferably, the terminal portion axis 66A is angled between about 90 degrees and about 140 degrees relative to the intermediate portion axis 60A, and more preferably is angled about 120 degrees. The terminal portion 64A includes cutting means for creating a remaining trabecular leaflet at, or just posterior of, Schwalbe's line in an eye.

The embodiment of the instrument 40A differs from the above-discussed first embodiment of the instrument 40 in that the terminal portion 64A is configured as a left-angled instrument relative to the hand grip 44A and the other portions of the tip 52A.

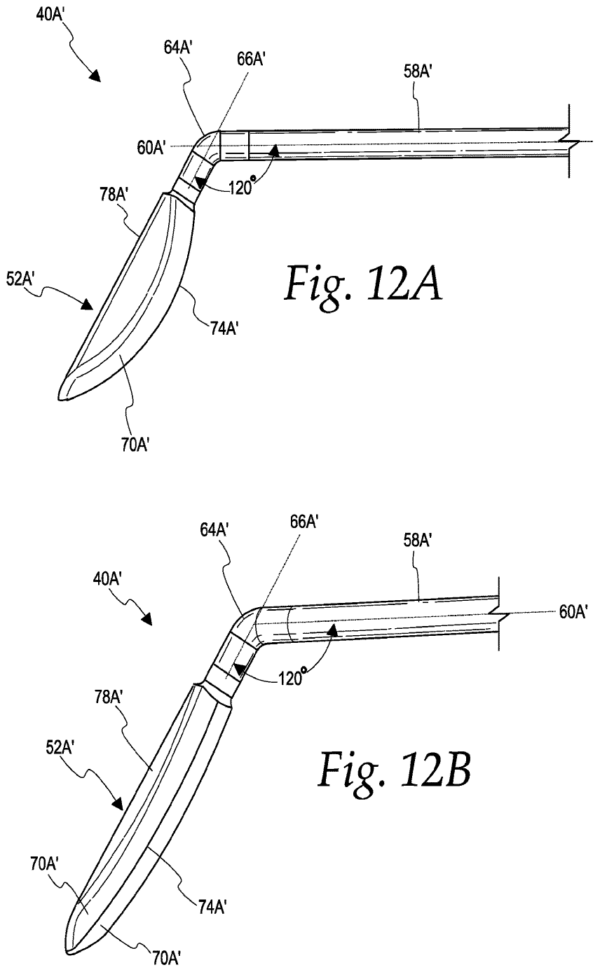

Referring now to FIGS. 12A and 12B, a variation of the second illustrated embodiment of an instrument of the present invention is illustrated and designated as 40A′. The numbered features of the instrument 40A′ are designated generally with the suffix “A” and are analogous to features of the aforementioned illustrated embodiment of the instrument 40A that share the same number (without the suffix letter “A′”). The instrument 40A′ operates in an identical manner as described in detail above and has the same basic features of a hand grip with a tip including a base portion that extends from the distal end of the hand grip and defines a base portion axis through its geometric center. The base portion axis is substantially parallel to, and coincident with, the central axis of the hand grip. The tip further includes an intermediate portion 58A′ extending from the base portion along an intermediate portion axis 60A′ that is transverse or angled relative to the base portion axis, Preferably, the intermediate portion axis 60A′ is angled between about 130 degrees and about 160 degrees relative to the base portion axis, and more preferably angled about 145 degrees.

The tip 52A′ includes a terminal portion 64A′ extending from the intermediate portion 58A′ along a terminal portion axis 66A′ that is transverse to the intermediate portion axis 56A′. The terminal portion 64A′ includes cutting means for creating a remaining trabecular leaflet at, or just posterior of, Schwalbe's line in an eye.

The embodiment of the instrument 40A′ differs from the above-discussed second embodiment of the instrument 40A in that the terminal portion axis 66A′ is angled about 120 degrees to the intermediate portion axis 60A′. The inventors have found that increasing this angle greatly enhances the ability of a variety of users to create the aforementioned leaflet in a left-angled instrument (as illustrated), a right-angled instrument (e.g., as shown with embodiment of the instrument 40 in FIGS. 1-6), or a straight, non-angled instrument where the distal portion of the instrument extends in a plane containing the central axis of the hand grip.

The arcuate cutting edge 74A′ is angled about between about 30 degrees and about 50 degrees out of the plane containing the axes 60A′ and 66A′, and more preferably about 40 degrees out of the plane.

With reference now to FIG. 14 additional embodiments of systems of the present invention are diagrammatically illustrated and designated as 40B with associated equipment (such as power sources, vacuum and irrigation fluid supply, conduits, cords, control hardware and software, etc.) designated as 200B. While the preferred form of the present system is a handheld, manually operated cutting instrument with one or more cutting edges or surfaces to create the trabecular leaflet at, or just posterior of, Schwalbe's line in an eye as described above, the inventors have found that other systems may be used to create a functionally similar leaflet.

In one example, the system 40B includes a handheld invasive laser probe that is inserted through the cornea into an area adjacent the trabecular meshwork such as the instruments described in U.S. Pat. Nos. 4,846,172 A and 6,241,721 B1, or other commercially available laser probe surgical systems, which may incise the trabecular meshwork with focused energy in the form of Neodymium: yttrium-aluminum-garnet (Nd:Yag) laser, excimer laser, femtosecond laser, green light laser, diode laser, or other commercially available ophthalmic or medical laser beams. The operation of such laser probe systems and the associated laser generation equipment and associated control software is known in the art and is encompassed generally by element 200B in FIG. 14. The operative or distal end of the laser probe of the system 40B must be brought in proximity to the trabecular meshwork and may cut the meshwork to create a trabecular leaflet 150 at, or just posterior of, Schwalbe's line in the eye. The energy of the laser beam and/or the path and extent of the incision may be manually adjusted by the surgeon or may be controlled automatically with associated equipment and control systems having servo motors or stepper motors. It will be understood that, unlike the mechanical surgical instruments of the present invention, the use of an invasive laser energy probe presents unique difficulties and increased capital equipment costs. For example, non-targeted, surrounding tissues proximate to the trabecular leaflet 150 may potentially be damaged by the laser energy. Furthermore, the laser probe may not be as maneuverable within the eye as a sharpened microsurgical cutting instrument as described above and may require one or more specialized tip portions to create the requisite angled cleavage of the trabecular leaflet 150.

In another example, the system 40B includes a non-invasive laser instrument that is located outside of the cornea and targeted at the trabecular meshwork, with or without an associated lens or mirror located on, or adjacent to, the cornea, such as the instruments described in U.S. Pat. Nos. 6,059,772 A, 10,821,023 B2, and 11,039,958 B2, and U.S. Patent Application Publication Nos. US 2020/01468871 A1 and US 2024/0058169 A1, which are all incorporated herein in their entireties, or other commercially available laser systems, which may incise the trabecular meshwork with focused energy in the form of Neodymium: yttrium-aluminum-garnet (Nd:Yag) laser, excimer laser, femtosecond laser, green light laser, diode laser, or other commercially available ophthalmic or medical laser beams. The operation of such laser systems and the associated laser generation equipment and associated control software is known in the art and is encompassed generally by element 200B in FIG. 14. The operative or distal end of the laser instrument of the system 40B must be directed through the cornea, either directly or via a lens or mirror positioned proximate the cornea, and may direct a focused laser into the anterior chamber of the eye to incise the meshwork to create a trabecular leaflet 150 at, or just posterior of, Schwalbe's line in an eye. The energy of the laser beam and/or the path and extent of the incision may be manually adjusted by the surgeon or may be controlled automatically with associated equipment and control systems. It will be understood that, unlike the mechanical surgical instruments of the present invention, the use of a non-invasive laser energy instrument presents unique difficulties and increased capital equipment costs. For example, non-targeted, surrounding tissues of the cornea or the tissues proximate to the trabecular leaflet 150 may potentially be damaged by the laser energy. Furthermore, the laser beam may not be as maneuverable within the eye as a sharpened microsurgical cutting instrument as described above. The system 40B may further be provided with an optical coherence tomography (OCT) beam configured to create an image of the eye. The image of the eye may determine the location and/or extent of the trabecular leaflet treatment or formation within the eye. Such OCT imaging systems and control methods are discussed in U.S. Pat. No. 10,821,023 B2 and US 2024/0058169 A1. Alternatively, the formation of the trabecular leaflet may be visualized through a gonio lens. OCT imaging systems, non-invasive lasers, and control systems are described in US 2021/0220176 A1, which is incorporated herein by reference in its entirety. OCT systems and components are commercially available from vendors, such as Optovue Inc., Fremont, Calif., Topcon Medical Systems, Oakland, N J, Carl Zeiss Meditec A G, Germany, Nidek, Aichi, Japan, Thorlabs, Newton, N. J., Santec, Aichi, Japan, and Axsun, Billercia, M A. Non-invasive lasers are commercially available from vendors such as Newport, Irvine, Calif., Coherent, Santa Clara, Calif., Amplitude Systems, Pessac, France, and NKT Photonics, Birkerod, Denmark. Combined imaging and laser systems are commercially available from vendors such as Vialase, Aliso Viejo, Calif. and Belkin Vision, Yavne, Israel. Unlike these commercially available systems, which vaporize or ablate a relatively large volume of meshwork in a short arc (relative to the optical axis), the present invention utilizes a narrow, linear incision to form a trabecular flap or leaflet along a relatively longer arc (relative to the optical axis).

In still another example, the system 40B includes a vibratory cutting Instrument having at least one vibratory cutting edge or operative end which is inserted through the cornea and brought proximate to the trabecular meshwork. The vibratory instrument may be, for example, a longitudinally vibrating phacoemulsification handpiece, a torsionally-vibrating phacoemulsification handpiece, an elliptically vibrating phacoemulsification handpiece, a phacoemulsification handpiece configured for vibratory movement in three dimensions, a vitrectomy handpiece, a piezo electric handpiece, an ultrasound handpiece, a solenoid valve handpiece, pneumatic, or a battery powered handpiece. Other commercially available vibratory handpieces may be used with system 40B disclosed herein, which may be suitable to incise the trabecular meshwork as set forth above. The operation of such vibratory cutting systems and the associated vibration generation equipment and associated control software is known in the art and is encompassed generally by element 200B in FIG. 14. The operative or distal end of the vibratory cutting instrument of the system 40B must be brought in proximity to the meshwork to create a trabecular leaflet 150 at, or just posterior of, Schwalbe's line in an eye. The energy of the vibratory instrument and/or the path and extent of the incision may be manually adjusted by the surgeon or may be controlled automatically with associated equipment and control systems. It will be understood that, unlike the manually-operated mechanical surgical instruments of the present invention discussed above, the use of a vibratory instrument presents unique difficulties and increased capital equipment costs. For example, non-targeted, surrounding tissues of the tissues proximate to the trabecular leaflet 150 may potentially be damaged by the vibratory cutting edge of the instrument. Furthermore, the vibratory cutting instrument may not be as maneuverable within the eye as a manual, sharpened microsurgical cutting instrument as described above.

In yet another example, the system 40B includes a cautery, thermal energy, or cryo cutting instrument such as the instruments described in U.S. Pat. Nos. 6,979,328 B2 and 11,464,669 B2, which are incorporated herein in their entireties, such instruments being inserted through the cornea and brought proximate to the trabecular meshwork. Other commercially available surgical instruments may be used with system 40B disclosed herein, which may be suitable to incise the trabecular meshwork as set forth above. The operation of such cutting systems and the associated thermal or cryo generation equipment and associated control software is known in the art and is encompassed generally by element 200B in FIG. 14. The operative or distal end of the cutting instrument of the system 40B must be brought in proximity to the meshwork to create a trabecular leaflet 150 at, or just posterior of, Schwalbe's line in an eye. The energy of the instrument and/or the path and extent of the incision may be manually adjusted by the surgeon or may be controlled automatically with associated equipment and control systems. It will be understood that, unlike the manually-operated mechanical surgical instruments of the present invention discussed above, the use of a such instruments presents unique difficulties and increased capital equipment costs. For example, non-targeted, surrounding tissues of the tissues proximate to the trabecular leaflet 150 may potentially be damaged by the use of such instruments. Furthermore, such instruments may not be as maneuverable within the eye as a manual, sharpened microsurgical cutting instrument as described above.

Generally, the systems and instruments illustrated and discussed herein may include one or more through passages communicating with an irrigation fluid supply source (either located in a reservoir in the hand grip, or located in an external pressurized container or machine and connected to the hand grip through tubing). The hand grip may include a pressure switch, bellows, or other means to facilitate the selective application of the irrigation fluid from the reservoir or irrigation fluid supply source to a target surgical site or location at the distal, operative end of the instrument proximate the cutting means. Such a hand grip with a reservoir and pressure switch is disclosed in International Application Publication No. WO/2023/018568 of Nallakrishnan, which is incorporated by reference herein in its entirety. Alternatively, an irrigation fluid may be supplied via a sleeve or tube situated around a portion of the instrument. There are many commercially available irrigating handpieces or systems on the market, and it will be understood that the instrument may be adapted to function with such handpieces or systems.

Various modifications and alterations to this invention will become apparent to those skilled in the art without departing from the scope and spirit of this invention. Illustrative embodiments and examples are provided as examples only and are not intended to limit the broadest scope of the present invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. A method of using a goniotomy surgical system to treat the trabecular meshwork of the eye, the method comprising the steps of:
obtaining a goniotomy surgical system including a means for creating a trabecular leaflet at Schwalbe's line in an eye, said trabecular leaflet characterized by an opening angle of between about 4 degrees and about 45 degrees defined between said trabecular leaflet and the outer wall of the Schlemm's Canal; and
creating a trabecular leaflet in the eye with said means, said trabecular leaflet characterized by an opening angle of between about 4 degrees and about 45 degrees defined between said trabecular leaflet and the outer wall of the Schlemm's Canal.

14. The method of using a goniotomy surgical system to treat the trabecular meshwork of the eye in accordance with claim 13, wherein said trabecular leaflet is characterized by an opening angle of about 10 degrees defined between said trabecular leaflet and the outer wall of the Schlemm's Canal.

15. The method of using a goniotomy surgical system to treat the trabecular meshwork of the eye in accordance with claim 13, wherein said trabecular leaflet is further characterized as having an arcuate, concave portion facing the outer wall of the Schlemm's Canal.

16. The method of using a goniotomy surgical system to treat the trabecular meshwork of the eye in accordance with claim 15, wherein said arcuate, concave portion of said trabecular leaflet defines a central angle of between 30 degrees and 60 degrees.

17. The method of using a goniotomy surgical system to treat the trabecular meshwork of the eye in accordance with claim 15, wherein said arcuate, concave portion of said trabecular leaflet defines a ratio of arc length to radius of between about 0.5 and 0.9.

18. The method of using a goniotomy surgical system to treat the trabecular meshwork of the eye in accordance with claim 13, wherein said means for creating a trabecular leaflet is a handheld microsurgical cutting instrument having at least one sharpened edge.

19. The method of using a goniotomy surgical system to treat the trabecular meshwork of the eye in accordance with claim 13, wherein said means for creating a trabecular leaflet is an invasive laser cutting instrument for being inserted into the anterior chamber of the eye and having at least one laser source.

20. The method of using a goniotomy surgical system to treat the trabecular meshwork of the eye in accordance with claim 13, wherein said means for creating a trabecular leaflet is a non-invasive laser cutting instrument configured to form said trabecular leaflet from outside of the cornea of the eye and having at least one laser source.

21. The method of using a goniotomy surgical system to treat the trabecular meshwork of the eye in accordance with claim 13, wherein said means for creating a trabecular leaflet is a vibratory cutting instrument configured to form said trabecular leaflet having at least one vibratory cutting edge.

22. The method of using a goniotomy surgical system to treat the trabecular meshwork of the eye in accordance with claim 13, wherein said means for creating a trabecular leaflet is a thermal or cryo cutting instrument configured to form said trabecular leaflet and having at least one thermal or cryo cutting tip.

23. The method of using a goniotomy surgical system to treat the trabecular meshwork of the eye in accordance with claim 13, wherein said system further comprises one of an internal reservoir of an irrigating fluid or a connection to an external reservoir of irrigating fluid and means for selectively applying said irrigating fluid to said trabecular leaflet.

24. The method of using a goniotomy surgical system to treat the trabecular meshwork of the eye in accordance with claim 13, wherein the step of creating a trabecular leaflet in the eye with said means further comprises creating said trabecular leaflet between about 90 degrees and about 180 degrees around the periphery of the eye relative to the optical axis of the eye.

25. The method of using a goniotomy surgical system to treat the trabecular meshwork of the eye in accordance with claim 24, wherein the step of creating a trabecular leaflet in the eye with said means further comprises creating said trabecular leaflet about 120 degrees around the periphery of the eye relative to the optical axis of the eye.

26. The method of using a goniotomy surgical system to treat the trabecular meshwork of the eye in accordance with claim 13, wherein said system further comprises an imaging system having an optical coherence tomography beam configured to create an image of the eye; and said method comprises the further step of controlling said means for creating a trabecular leaflet based on said image of the eye.

27. (canceled)

28. (canceled)
